(19) United States
(12) Patent Application Publication
(10) Pub. No.: US 2022/0004899 A1
(43) Pub. Date: Jan. 6, 2022

(54) METHOD FOR DIAGNOSING EXCEPTION DATA, USER TERMINAL APPARATUS AND CLOUD SERVER

(71) Applicant: Samsung Electronics Co., Ltd., Suwon-si (KR)

(72) Inventors: Bo HU, Xi"ian (CN); Sanqi LI, Xi"ian (CN); Chunmiao JIANG, Xi"ian (CN)

(73) Assignee: Samsung Electronics Co., Ltd., Suwon-si (KR)

(21) Appl. No.: 17/010,246

(22) Filed: Sep. 2, 2020

(30) Foreign Application Priority Data
Jul. 6, 2020 (CN) 2020 1 0641821.9

(51) Int. Cl. G06N 5/04; G06N 20/00

(52) U.S. Cl. CPC G06N 5/04; G06N 20/00

(57) ABSTRACT

A user terminal apparatus includes an alarm; a collector including one or more sensors configured to collect characteristic data of a target object; and a controller configured to obtain a terminal-side exception judgment result by inputting the characteristic data into a terminal-side exception diagnosis model, in response to the terminal-side exception judgment result indicating that the characteristic data is abnormal, transmit the characteristic data to a cloud server, receive diagnostic information from the cloud server, and based on the received diagnostic information, update at least one of a terminal-side multi-classification model or the terminal-side exception diagnosis model, or control the alarm to issue an alarm signal indicating that the characteristic data is abnormal.

### CROSS-REFERENCE TO RELATED APPLICATIONS

A claim for priority under 35 U.S.C. § 119 is made to Chinese Patent Application No. 202010641821.9 filed on Jul. 6, 2020, in the State Intellectual Property Office (SIPO), the entire contents of which are hereby incorporated by reference.

### BACKGROUND

### 1. Field

The present disclosure generally relates to a field of computer technology, and more particularly, relates to a method for diagnosing exception data, a user terminal apparatus and a cloud server.

### 2. Description of Related Art

With the continuous progress of science and technology, artificial intelligence has been widely studied and applied. A core of artificial intelligence is machine learning, which is a fundamental way to make computers intelligent. Nowadays, machine learning is widely used in many fields, for example, medical diagnostics, biometrics recognition, data mining, computer vision, natural language processing, search engines, credit card fraud detection, speech and handwriting recognition, robotics applications, and so on.

However, in widely using machine learning models, a machine learning model trained offline sometimes cannot meet needs of online services. For example, with an elapse of time, prediction data in an application of the machine learning model may be different from training data used in developing and training the machine learning model, so that prediction results are not accurate. Therefore, it is often necessary to continuously update the machine learning model during the application. However, there is currently no particularly good update method, and update process is usually triggered manually by a user, and update efficiency is usually low. In addition, when the machine learning model is used to predict the data to be predicted, a prediction result will, in at least some cases, be always output, and thus real abnormally predicted data can be difficult to detect in a timely and effective manner.

### SUMMARY

At least some example embodiments of the inventive concepts provide a method for diagnosing exception data, a user terminal apparatus and a cloud server, to overcome defect that existing data identification methods cannot process data to be predicted in a new environment.

According to at least some example embodiments of the inventive concepts, a user terminal apparatus includes an alarm; a collector including one or more sensors configured to collect characteristic data of a target object; and a controller configured to obtain a terminal-side exception judgment result by inputting the characteristic data into a terminal-side exception diagnosis model, in response to the terminal-side exception judgment result indicating that the characteristic data is abnormal, transmit the characteristic data to a cloud server, receive diagnostic information from the cloud server, and based on the received diagnostic information, update the terminal-side exception diagnosis model, or control the alarm to issue an alarm signal indicating that the characteristic data is abnormal based on the diagnostic information.

According to at least some example embodiments of the inventive concepts, a method for diagnosing exception data includes collecting characteristic data of a target object; obtaining a terminal-side exception judgment result by inputting the characteristic data into a terminal-side exception diagnosis model; in response to the terminal-side exception judgment result indicating that the characteristic data is abnormal, transmitting the characteristic data to a cloud server; receiving diagnostic information from the cloud server; and based on the diagnostic information, updating the terminal-side exception diagnosis model, or issuing an alarm signal indicating that the characteristic data is abnormal.

According to at least some example embodiments of the inventive concepts, cloud server includes memory storing computer-executable instructions; and one or more processors configured to execute the computer-executable instructions such that the or more processors are configured to receive characteristic data of a target object from a user terminal apparatus, obtain a cloud-side exception judgment result by inputting the characteristic data into a cloud-side exception diagnosis model, obtain diagnostic information of the characteristic data according to the cloud-side exception judgment result; and transmit the diagnostic information to the user terminal apparatus.

According to at least some example embodiments of the inventive concepts, a computer-readable storage medium storing a computer program that, when executed by a processor, causes the processor to perform the method for diagnosing exception data as described above is provided.

According to the method for diagnosing the exception data, the user terminal apparatus, and the cloud server according to at least some example embodiments of the inventive concepts, a defect that the user terminal apparatus cannot quickly adapt or cannot quickly issue an alarm when characteristic data of a target object in an actual scene is offset can be solved.

### BRIEF DESCRIPTION OF THE DRAWINGS

The above and other features and advantages of example embodiments of the inventive concepts will become more apparent by describing in detail example embodiments of the inventive concepts with reference to the attached drawings. The accompanying drawings are intended to depict example embodiments of the inventive concepts and should not be interpreted to limit the intended scope of the claims. The accompanying drawings are not to be considered as drawn to scale unless explicitly noted.

FIG. 1 illustrates a block diagram of a user terminal apparatus according to at least some example embodiments of the inventive concepts;

FIG. 2 illustrates a flowchart of a method for diagnosing exception data according to at least some example embodiments of the inventive concepts;

FIG. 3 illustrates a block diagram of a cloud server according to at least some example embodiments of the inventive concepts;

FIG. 4 illustrates a flowchart of a method for diagnosing exception data according to at least one example embodiment of the inventive concepts.

### DETAILED DESCRIPTION OF EXEMPLARY EMBODIMENTS

As is traditional in the field of the inventive concepts, embodiments are described, and illustrated in the drawings, in terms of functional blocks, units and/or modules. Those skilled in the art will appreciate that these blocks, units and/or modules are physically implemented by electronic (or optical) circuits such as logic circuits, discrete components, microprocessors, hard-wired circuits, memory elements, wiring connections, and the like, which may be formed using semiconductor-based fabrication techniques or other manufacturing technologies. In the case of the blocks, units and/or modules being implemented by microprocessors or similar, they may be programmed using software (e.g., microcode) to perform various functions discussed herein and may optionally be driven by firmware and/or software. Alternatively, each block, unit and/or module may be implemented by dedicated hardware, or as a combination of dedicated hardware to perform some functions and a processor (e.g., one or more programmed microprocessors and associated circuitry) to perform other functions. Also, each block, unit and/or module of the embodiments may be physically separated into two or more interacting and discrete blocks, units and/or modules without departing from the scope of the inventive concepts. Further, the blocks, units and/or modules of the embodiments may be physically combined into more complex blocks, units and/or modules without departing from the scope of the inventive concepts.

Reference will now be made in detail to at least some example embodiments of the inventive concepts, examples of which are illustrated in the accompanying drawings, wherein like reference numerals refer to like elements throughout. Hereinafter, at least some example embodiments of the inventive concepts will be explained with reference to the accompany drawings so as to explain the present disclosure.

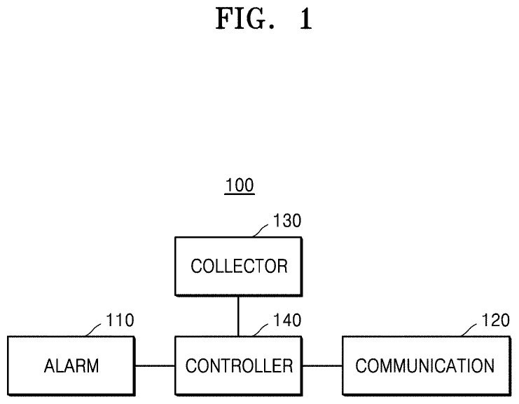

FIG. 1 illustrates a block diagram of a user terminal apparatus according to at least some example embodiments of the inventive concepts. Here, as an example, the user terminal apparatus may be an electronic apparatus with a machine learning function, such as a wearable device, a personal computer, a smart phone, and so on.

As shown in FIG. 1, the user terminal apparatus 100 according to at least one example embodiment of the inventive concepts includes an alarm 110, a communicator 120, a collector 130 and a controller 140.

In particular, the communicator 120 is configured to communicate with a cloud server 200.

The collector 130 is configured to collect characteristic data of a target object.

For example, the collector 130 may collect physical characteristic data of a user for medical diagnosis, such as heart rate, blood pressure, blood glucose concentration, diastolic blood pressure, age, etc. For example, the collector 130 may include hardware such as one or more sensors, for example, in accordance with known wearable monitors (e.g., heart rate/blood pressure/blood glucose monitors) that include sensors for collecting various health information. The collector 130 may also collect audio characteristic data of the user (e.g., the user's voice) for speech recognition. For example, the collector 130 may include audio detecting sensors such as one or more microphones. Here, it should be understood that the characteristic data of the target object which can be collected by the collector 130 is not limited to the above examples, and may also be other characteristic data of the target object. No limitation is made here in the present disclosure. The controller 140 will now be discussed.

According to at least some example embodiments of the inventive concepts, the controller 140 may be, or include, processing circuitry. The processing circuitry of the controller 140 may include one or more circuits or circuitry (e.g., hardware) specifically structured to carry out and/or control some or all of the operations described in the present disclosure as being performed by the controller 140 or the user terminal apparatus 100 (or an element thereof). According to at least one example embodiment of the inventive concepts, the processing circuitry of the controller 140 may include memory and one or more processors executing computer-readable code (e.g., software and/or firmware) that is stored in the memory and includes instructions for causing the one or more processors to carry out and/or control some or all of the operations described in the present disclosure as being performed by the controller 140 or the user terminal apparatus 100 (or an element thereof). According to at least one example embodiment of the inventive concepts, the processing circuitry of the controller 140 may include, for example, a combination of the above-referenced hardware and one or more processors executing computer-readable code.

According to at least some example embodiments of the inventive concepts, the controller 140 inputs the characteristic data into a terminal-side exception diagnosis model to obtain a terminal-side exception judgment result.

As an example, the terminal-side exception diagnosis model may be a binary classifier, the binary classifiers can determine whether the input characteristic data is exception data based on the characteristic data, and obtain a terminal-side exception judgment result indicating whether the characteristic data is the exception data.

For example, in practical applications, there may be many reasons why the characteristic data can be judged as exception data. For example, the binary classifier (i.e., the terminal-side exception diagnosis model) provided in the user terminal apparatus 100 may drift over time, or there may be a huge difference between the characteristic data and training data used during a training process. Therefore, in order to determine whether the characteristic data is really abnormal, the user terminal apparatus 100 may send that characteristic data to the cloud server 200 son the cloud server 200 may determine the cause of the user terminal apparatus 100 identifying the characteristic data as abnormal.

In particular, when the terminal-side exception judgment result indicates that the characteristic data is abnormal, the controller 140 may send the characteristic data to the cloud server 200 by controlling the communicator 120 to transmit the characteristic data to the cloud server 200, and the controller 140 may receive the diagnostic information from the cloud server 200 by controlling the communicator 120 to receive the diagnostic information. In this way, the user terminal apparatus 100 may use the cloud server 200 to determine whether the interpretation of the characteristic data as exception data by the user terminal apparatus 100 is accurate.

After receiving the diagnostic information, the controller 140 updates the terminal-side multi-classification model and/or the terminal-side exception diagnosis model based on the diagnostic information, or controls the alarm 110 to issue an alarm signal indicating that the characteristic data is abnormal based on the diagnostic information.

As an example, the terminal-side multi-classification model may be a multi-classifier, and the multi-classifier may perform characteristic classification on the characteristic data. For example, when the characteristic data is input into the terminal-side multi-classification model, the terminal-side multi-classification model may output a category to which the characteristic data belongs. For example, if the characteristic data includes pregnancy times, blood glucose concentration, diastolic blood pressure, family medical history and age of a female, when the characteristic data is input into the terminal-side multi-classification model, the terminal-side multi-classification model can determine whether the female has diabetes according to the pregnancy times, the blood glucose concentration, the diastolic blood pressure, the family medical history and the age of the female.

In particular, in one example, if the diagnostic information indicates that a cloud-side exception judgment result of the cloud server 200 is different from the terminal-side exception judgment result, the controller 140 receives a new terminal-side multi-classification model and a new terminal-side exception diagnosis model from the cloud server 200 (e.g., by controlling the communicator 120 to receive the new terminal-side multi-classification model and the new terminal-side exception diagnosis model from the cloud server 200), and the controller 140 updates the current terminal-side multi-classification model to the new terminal-side multi-classification mode and updates the current terminal-side exception diagnosis model to the new terminal-side exception diagnosis model. In this way, the terminal-side multi-classification model and/or the current terminal-side exception diagnosis model can be updated, such that the terminal-side multi-classification model and/or terminal-side exception diagnosis model can adapt to the characteristic data of the target object in a new environment.

In another case, if the diagnostic information indicates that the cloud-side exception judgment result of the cloud server is the same as the terminal-side exception judgment result, the controller 140 issues an alarm indicating that the characteristic data is abnormal (e.g., by controlling the alarm 110 to issue the alarm signal indicating that the characteristic data is abnormal).

In particular, in response to the diagnostic information indicating that the cloud-side exception judgment result of the cloud server is the same as the terminal-side exception judgment result, the user terminal apparatus 100 may determine that the characteristic data is indeed significantly different from the characteristic data of a previous target object and that the user needs to pay attention to it. At this time, the alarm 110 can issue an alarm to inform the user that the characteristic data of the user is abnormal, thereby warning the user to handle the exception immediately.

Using the user terminal apparatus according to at least one example embodiment of the inventive concepts, a defect that the user terminal apparatus cannot quickly adapt or cannot quickly issue an alarm when the characteristic data of the target object in an actual scene is offset can be solved.

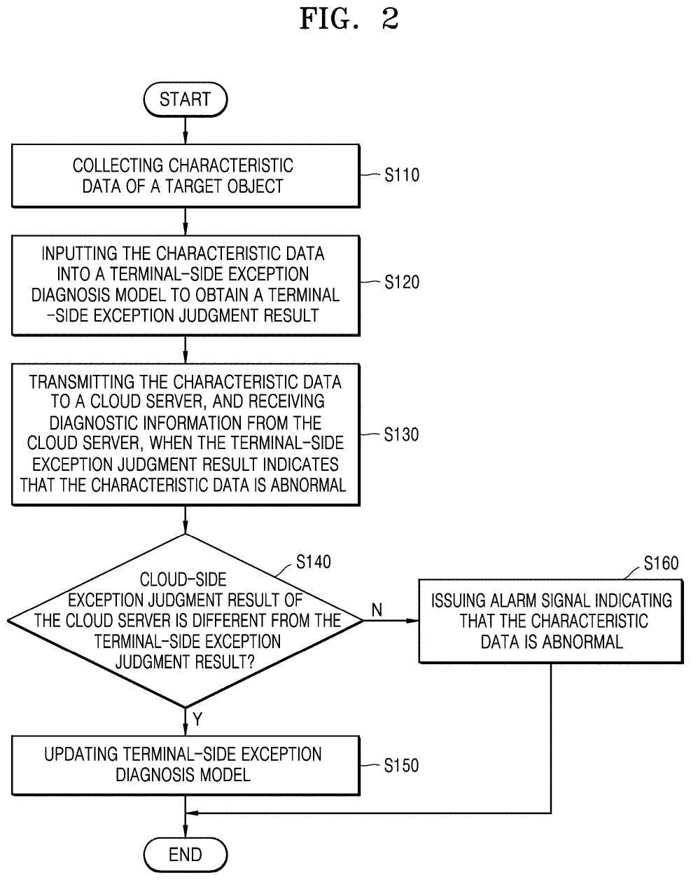

FIG. 2 illustrates a flowchart of a method for diagnosing exception data according to at least some example embodiments of the inventive concepts.

As shown in FIG. 2, at step S110, characteristic data of a target object is collected.

For example, physical characteristic data of the user for medical diagnosis, such as heart rate, blood pressure, blood glucose concentration, diastolic blood pressure, age, etc., can be collected. Audio characteristic data of the user for speech recognition also can be collected. Here, it should be understood that the characteristic data of the target object which can be collected is not limited to the above examples, and may also be other characteristic data of the target object. No limitation is made here in the present disclosure.

At step S120, the characteristic data is inputted into a terminal-side exception diagnosis model to obtain a terminal-side exception judgment result.

As an example, the terminal-side exception diagnosis model may be a binary classifier, the binary classifiers can determine whether the input characteristic data is exception data based on the characteristic data, and obtain a terminal-side exception judgment result indicating whether the characteristic data is the exception data.

In particular, in practical applications, there are many causes why the characteristic data is judged as the exception data. For example, the binary classifier (i.e., a terminal-side exception diagnosis model) provided in a user terminal apparatus 100 may drift over time, or there may be a huge difference between the characteristic data and the training data used during a training process. Therefore, in order to determine whether the characteristic data is really abnormal, the cause of the characteristic data being identified as abnormal by the user terminal apparatus 100 may be determined by the cloud server 200.

At step S130, the characteristic data is transmitted to the cloud server 200, and diagnostic information is received from the cloud server 200, when the terminal-side exception judgment result indicates that the characteristic data is abnormal. For example, the user terminal apparatus 100 may transmit the characteristic data to the cloud server 200 in response to the terminal-side exception judgment result indicating that the characteristic data is abnormal. Further, the cloud server 200 may respond by sending the user terminal apparatus 100 diagnostic information, and the user terminal apparatus 100 may receive the diagnostic information from the cloud server 200. Accordingly, the user terminal apparatus 100 may use information from the cloud server 200 to determine whether the user terminal apparatus 100 accurately identified the characteristic data as abnormal.

As is discussed in greater detail below with reference to steps S140-S160, the terminal-side exception diagnosis model may be updated based on the diagnostic information, or an alarm signal indicating that the characteristic data is abnormal may be issued based on the diagnostic information. As another example, a terminal-side multi-classification model may be updated based on the diagnostic information.

As an example, the terminal-side multi-classification model may be a multi-classifier, and the multi-classifier may perform characteristic classification on the characteristic data. For example, when the characteristic data is input into the terminal-side multi-classification model, the terminal-side multi-classification model may output a category to which the characteristic data belongs. For example, if the characteristic data includes pregnancy times, blood glucose concentration, diastolic blood pressure, family medical history and age of a female, when the characteristic data is input into the terminal-side multi-classification model, the terminal-side multi-classification model can determine whether the female has diabetes according to the pregnancy times, the blood glucose concentration, the diastolic blood pressure, the family medical history and the age of the female. Steps S140-S160 will now be discussed below.

At step S140, it is determined whether the diagnostic information indicates that a cloud-side exception judgment result of the cloud server is different from the terminal-side exception judgment result. If the diagnostic information indicates that a cloud-side exception judgment result of the cloud server is different from the terminal-side exception judgment result, the method proceeds to step S150. At step S150, a new terminal-side exception diagnosis model is received from the cloud server 200, and the terminal-side exception diagnosis model (i.e., the current terminal-side exception diagnosis model) is updated to the new terminal-side exception diagnosis model. As another example, in step S150, instead of (or, alternatively, in addition to) receiving the new terminal-side exception diagnosis model and updating the terminal-side exception diagnosis model, a new terminal-side multi-classification model may be received from the cloud server 200, and accordingly, the terminal-side multi-classification model (i.e., the current terminal-side multi-classification model) may be updated to the new terminal-side multi-classification model. In this way, the terminal-side multi-classification model and/or terminal-side exception diagnosis model can be updated, such that the terminal-side multi-classification model and/or terminal-side exception diagnosis model can adapt to the characteristic data of the target object in the new environment.

In another case, if the diagnostic information indicates that the cloud-side exception judgment result of the cloud server is the same as the terminal-side exception judgment result, at step S160, an alarm signal indicating that the characteristic data is abnormal is issued.

In particular, if the diagnostic information indicates that the cloud-side exception judgment result of the cloud server is the same as the terminal-side exception judgment result, it indicates that the characteristic data is indeed significantly different from characteristic data of a previous target object and the user need to pay attention to it. At this time, an alarm can be issued to inform the user that the characteristic data of the user is abnormal, thereby warning the user to handle the exception immediately.

Using the method for diagnosing the exception data according to at least one example embodiment of the inventive concepts, a defect that the user terminal apparatus cannot quickly adapt or cannot quickly issue an alarm when the characteristic data of the target object in an actual scene is offset can be solved.

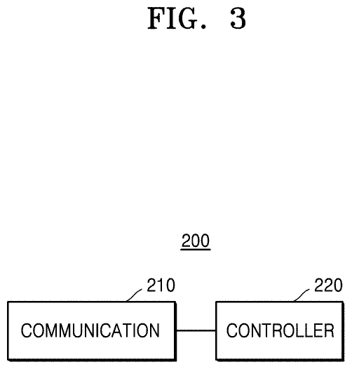

FIG. 3 illustrates a block diagram of a cloud server according to at least some example embodiments of the inventive concepts.

As shown in FIG. 3, the cloud server 200 according to at least some example embodiments of the inventive concepts includes a communicator 210 and a controller 220.

According to at least some example embodiments of the inventive concepts, the controller 220 may be, or include, processing circuitry. The processing circuitry of the controller 220 may include one or more circuits or circuitry (e.g., hardware) specifically structured to carry out and/or control some or all of the operations described in the present disclosure as being performed by the controller 220 or the cloud server 200 (or an element thereof). According to at least one example embodiment of the inventive concepts, the processing circuitry of the controller 220 may include memory and one or more processors executing computer-readable code (e.g., software and/or firmware) that is stored in the memory and includes instructions for causing the one or more processors to carry out and/or control some or all of the operations described in the present disclosure as being performed by the controller 220 or the cloud server 200 (or an element thereof). According to at least one example embodiment of the inventive concepts, the processing circuitry of the controller 220 may include, for example, a combination of the above-referenced hardware and one or more processors executing computer-readable code.

In particular, the controller 220 controls the communicator 210 to receive characteristic data of a target object from a user terminal apparatus.

The controller 220 inputs the characteristic data into a cloud-side exception diagnosis model to obtain a cloud-side exception judgment result.

Here, the cloud-side exception diagnosis model may be a binary classifier, the binary classifiers can determine whether the input characteristic data is exception data based on the characteristic data, and obtain a cloud-side exception judgment result indicating whether the characteristic data is the exception data.

In particular, the controller 220 obtains diagnostic information of the characteristic data according to the cloud-side exception judgment result, and controls the communicator 210 to transmit the diagnostic information to the user terminal apparatus 100. Here, the diagnostic information can indicate whether the cloud-side exception judgment result is the same as the terminal-side exception judgment result.

As an example, in one case, if the cloud-side exception judgment result indicates that the characteristic data is normal, to indicate that the cloud-side exception judgment result is different from the terminal-side exception judgment result, to indicate that the characteristic data is not exception data through a more accurate judgment, and the terminal-side multi-classification model or a terminal-side exception diagnosis model in the user terminal apparatus 100 are offset over time or there is a huge difference between the characteristic data and training data used during a training process of the terminal-side multi-classification model or the terminal-side exception diagnosis model, the controller 220 inputs the characteristic data into a cloud-side multi-classification model to obtain a cloud-side classification result.

Here, the cloud-side classification model may include a multi-classifier for classifying the characteristic data. Here, it should be noted that the number of classification categories of the cloud-side multi-classification model in the cloud server 200 is much more than those of the terminal-side multi-classification model in the user terminal apparatus 100. For example, the terminal-side multi-classification model in the user terminal apparatus 100 can classify three categories, and the cloud-side multi-classification model in the cloud server 200 can classify one hundred categories, the controller 220 inputs the characteristic data into a cloud-side multi-classification model to obtain a cloud-side classification result.

Thus, the controller 220 may be further configured to transmit the cloud-side classification result to the user terminal apparatus 100 (e.g., by controlling the communicator 210 to transmit the cloud-side classification result to the user terminal apparatus 100). In this way, the user terminal apparatus 100 can use the characteristic data and the cloud-side classification result as new training data to train the terminal-side multi-classification model, and use the characteristic data as new training data to train the terminal-side exception diagnosis model, to complete updating of the terminal-side multi-classification model and/or the terminal-side exception diagnosis model, such that the terminal-side multi-classification model and the terminal-side exception diagnosis model can adapt to the characteristic data of the target object in a new environment.

The controller 220 trains the terminal-side multi-classification model in the cloud server based on the characteristic data and the cloud-side classification result to obtain the new terminal-side multi-classification model.

For example, the controller 220 adds the characteristic data and the cloud-side classification result into historical data used for training the terminal-side multi-classification model, and uses the historical data to re-train the terminal-side multi-classification model, to obtain the new terminal-side multi-classification model.

The controller 220 trains the terminal-side exception diagnosis model in the cloud server based on the characteristic data and the cloud-side exception judgment result, to obtain the new terminal-side exception diagnosis model.

For example, the controller 220 adds the characteristic data into historical data used for training the terminal-side exception diagnosis model, and use the historical data to re-train the terminal-side exception diagnosis model, to obtain the new terminal-side multi-classification model.

The controller 220 control the communicator to transmit the new terminal-side multi-classification model and the new terminal-side exception diagnosis model to the user terminal apparatus 100. In this way, the user terminal apparatus 100 can complete updating of the terminal-side multi-classification model and/or the terminal-side exception diagnosis model, such that the terminal-side multi-classification model and the terminal-side exception diagnosis model can adapt to the characteristic data of the target object in a new environment.

In another case, if the cloud-side exception judgment result is the same as the terminal-side exception judgment result, the controller 220 controls the communicator 210 to transmit diagnostic information indicating that the characteristic data is abnormal to the user terminal apparatus 100, to indicate that the characteristic data is indeed the exception data through a more accurate judgment.

Using the cloud server according to at least one example embodiment of the inventive concepts, a defect that the user terminal apparatus cannot quickly adapt or cannot quickly issue an alarm when the characteristic data of the target object in an actual scene is offset can be solved.

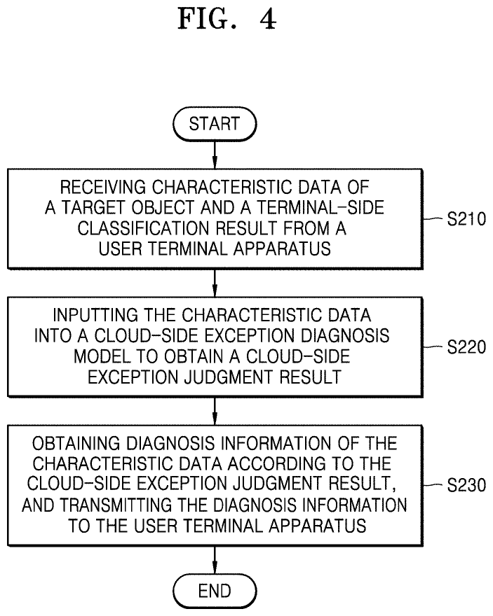

FIG. 4 illustrates a flowchart of a method for diagnosing exception data according to at least one example embodiment of the inventive concepts.

As shown in FIG. 4, at step S210, characteristic data of a target object and a terminal-side classification result is received from the user terminal apparatus 100.

At step S220, the characteristic data is inputted into a cloud-side exception diagnosis model to obtain a cloud-side exception judgment result.

Here, the cloud-side exception diagnosis model may be a binary classifier, the binary classifiers can determine whether the input characteristic data is exception data based on the characteristic data, and obtain a cloud-side exception judgment result indicating whether the characteristic data is the exception data.

At step S230, diagnostic information of the characteristic data is obtained according to the cloud-side exception judgment result, and the diagnostic information is transmitted to the user terminal apparatus 100.

As an example, in one case, if the cloud-side exception judgment result indicates that the characteristic data is normal, the characteristic data is inputted into a cloud-side multi-classification model to obtain the cloud-side classification result, the terminal-side multi-classification model in the cloud server 200 is trained based on the characteristic data and the cloud-side classification result to obtain the new terminal-side multi-classification model, the terminal-side exception diagnosis model in the cloud server is trained based on the characteristic data and the cloud-side exception judgment result to obtain a new terminal-side exception diagnosis model, the new terminal-side multi-classification model and the new terminal-side exception diagnosis model are transmitted to the user terminal apparatus 100.

In another case, if the diagnostic information indicates that a cloud-side exception judgment result of the cloud server is the same as the terminal-side exception judgment result, the diagnostic information indicating that the characteristic data is abnormal is transmit to the user terminal apparatus 100, to indicate that the characteristic data is indeed the exception data through a more accurate judgment.

Using the method for diagnosing the exception data according to at least one example embodiment of the inventive concepts, a defect that the user terminal apparatus cannot quickly adapt or cannot quickly issue an alarm when the characteristic data of the target object in an actual scene is offset can be solved.

The method for diagnosing the exception data according to at least one example embodiment of the inventive concepts can be implemented as computer program on a computer-readable storage medium The computer program when is executed by a processor, causes the processor to perform the method for diagnosing the exception data according to at least one example embodiment of the inventive concepts. The computer-readable storage medium is any data storage device that may store data which may be read out by a computer system. Examples of the computer-readable storage medium include: read-only memory, random-access memory, CD-ROMs, magnetic tapes, floppy disks, optical data storage devices and a carrier (such as data transmission via Internet through a wired or wireless transmission path).

Accordingly, the method for diagnosing the exception data, the user terminal apparatus and the cloud server according to at least some example embodiments of the inventive concepts can address the defect in which the user terminal apparatus cannot quickly adapt or cannot quickly issue an alarm when the characteristic data of the target object in the actual scene is offset.

Example embodiments of the inventive concepts having thus been described, it will be obvious that the same may be varied in many ways. Such variations are not to be regarded as a departure from the intended spirit and scope of example embodiments of the inventive concepts, and all such modifications as would be obvious to one skilled in the art are intended to be included within the scope of the following claims.

1. A user terminal apparatus, comprising:
an alarm;
a collector including one or more sensors configured to collect characteristic data of a target object; and
a controller configured to:
obtain a terminal-side exception judgment result by inputting the characteristic data into a terminal-side exception diagnosis model,
in response to the terminal-side exception judgment result indicating that the characteristic data is abnormal, transmit the characteristic data to a cloud server,
receive diagnostic information from the cloud server, and
based on the received diagnostic information,
update the terminal-side exception diagnosis model, or
control the alarm to issue an alarm signal indicating that the characteristic data is abnormal.

2. The user terminal apparatus of claim 1, wherein the controller is configured such that:
if the diagnostic information indicates that a cloud-side exception judgment result of the cloud server is different from the terminal-side exception judgment result,
the controller updates the terminal-side exception diagnosis model to a new terminal-side exception diagnosis model received from the cloud server.

3. The user terminal apparatus of claim 2, wherein the controller is configured such that:
if the diagnostic information indicates that a cloud-side exception judgment result of the cloud server is the same as the terminal-side exception judgment result,
the controller controls the alarm to issue the alarm signal indicating that the characteristic data is abnormal.

4. The user terminal apparatus of claim 3, wherein the characteristic data includes at least one of a heart rate, a blood pressure, and blood glucose concentration.

5. The user terminal apparatus of claim 3, wherein the one or more sensors include at least one microphone and the characteristic data includes audio characteristic data of a user's voice.

6. The user terminal apparatus of claim 1, wherein the characteristic data includes at least one of a heart rate, a blood pressure, and blood glucose concentration.

7. The user terminal apparatus of claim 1, wherein the controller is configured such that:
if the diagnostic information indicates that a cloud-side exception judgment result of the cloud server is different from the terminal-side exception judgment result,
the controller further updates a terminal-side multi-classification model to a new terminal-side multi-classification model received from the cloud server.

8. A cloud server, comprising:
memory storing computer-executable instructions; and
one or more processors configured to execute the computer-executable instructions such that the or more processors are configured to
receive characteristic data of a target object from a user terminal apparatus,
obtain a cloud-side exception judgment result by inputting the characteristic data into a cloud-side exception diagnosis model,
obtain diagnostic information of the characteristic data according to the cloud-side exception judgment result; and
transmit the diagnostic information to the user terminal apparatus.

9. The cloud server of claim 8, wherein the one or more processors are configured to execute the computer-executable instructions such that the or more processors are configured such that, if the cloud-side exception judgment result indicates that the characteristic data is normal, the one or more processors
train a terminal-side exception diagnosis model in the cloud server based on the characteristic data and the cloud-side exception judgment result to obtain a new terminal-side exception diagnosis model, and
transmit the new terminal-side exception diagnosis model to the user terminal apparatus.

10. The cloud server of claim 9, wherein the one or more processors are configured to execute the computer-executable instructions such that the one or more processors are configured such that, if the cloud-side exception judgment result indicates that the characteristic data is normal, the one or more processors
input the characteristic data into a cloud-side multi-classification model to obtain a cloud-side classification result,
train a terminal-side multi-classification model in the cloud server based on the characteristic data and the cloud-side classification result to obtain a new terminal-side multi-classification model,
transmit the new terminal-side multi-classification model to the user terminal apparatus.

11. The cloud server of claim 10, wherein the characteristic data includes at least one of a heart rate, a blood pressure, and blood glucose concentration.

12. The cloud server of claim 8, wherein the characteristic data includes at least one of a heart rate, a blood pressure, and blood glucose concentration.

13. The cloud server of claim 8, wherein the characteristic data includes audio characteristic data of a user's voice.

14. A method for diagnosing exception data, comprising:
collecting characteristic data of a target object;
obtaining a terminal-side exception judgment result by inputting the characteristic data into a terminal-side exception diagnosis model;
in response to the terminal-side exception judgment result indicating that the characteristic data is abnormal, transmitting the characteristic data to a cloud server;
receiving diagnostic information from the cloud server; and
based on the diagnostic information,
updating the terminal-side exception diagnosis model, or
issuing an alarm signal indicating that the characteristic data is abnormal.

15. The method of claim 14,
wherein updating the terminal-side exception diagnosis model includes, in response to the diagnostic information indicating that a cloud-side exception judgment result of the cloud server is different from the terminal-side exception judgment result,
updating the terminal-side exception diagnosis model to a new terminal-side exception diagnosis model received from the cloud server.

16. The method of claim 15,
wherein issuing the alarm signal indicating that the characteristic data is abnormal includes, in response to the diagnostic information indicating that a cloud-side exception judgment result of the cloud server is the same as the terminal-side exception judgment result,
issuing the alarm signal indicating that the characteristic data is abnormal.

17. The method of claim 14, further comprising:
in response to the diagnostic information indicating that a cloud-side exception judgment result of the cloud server is different from the terminal-side exception judgment result, updating a terminal-side multi-classification model to a new terminal-side multi-classification model received from the cloud server.

18. The method of claim 14, wherein the characteristic data includes at least one of a heart rate, a blood pressure, and blood glucose concentration.

19. The method of claim 14, wherein the characteristic data includes audio characteristic data of a user's voice.

20. A computer-readable storage medium storing a computer program that, when executed by a processor, causes the processor to perform the method of claim 15.

21.-22. (canceled)
